(19) United States
(12) Patent Application Publication
(10) Pub. No.: US 2009/0198121 A1
(43) Pub. Date: Aug. 6, 2009

(54) METHOD AND APPARATUS FOR COORDINATING CONTRAST AGENT INJECTION AND IMAGE ACQUISITION IN C-ARM COMPUTED TOMOGRAPHY

(76) Inventor: Martin Hoheisel, Erlangen (DE)

Correspondence Address: SCHIFF HARDIN, LLP;PATENT DEPARTMENT, 6600 SEARS TOWER, CHICAGO, IL, 60606-6473, US

(21) Appl. No.: 12/024,588

(22) Filed: Feb. 1, 2008

(51) Int. Cl. A61B 5/05

(52) U.S. Cl. 600/410; 600/425

(57) ABSTRACT

In a medical imaging system and method, an image data acquisition system is operable in a fluoroscopy mode to obtain a fluoroscopic image of a subject and is operable in a CT mode to obtain projection data sets of the subject that are used to reconstruct a three-dimensional image of a region of interest of the subject. For imaging procedures involving the administration of contrast agent to the subject, the image data acquisition apparatus is operated in the fluoroscopy mode at the time the administration of contrast agent is begun, and the filling of vessels with the contrast agent is automatically monitored by fluoroscopy. When the automatic monitoring indicates that an optimal degree of filling of the vessels with the contrast agent has occurred, the image data acquisition apparatus is automatically switched to operate in the CT mode to acquire projection data sets of the region of interest containing the vessels filled with contrast agent.

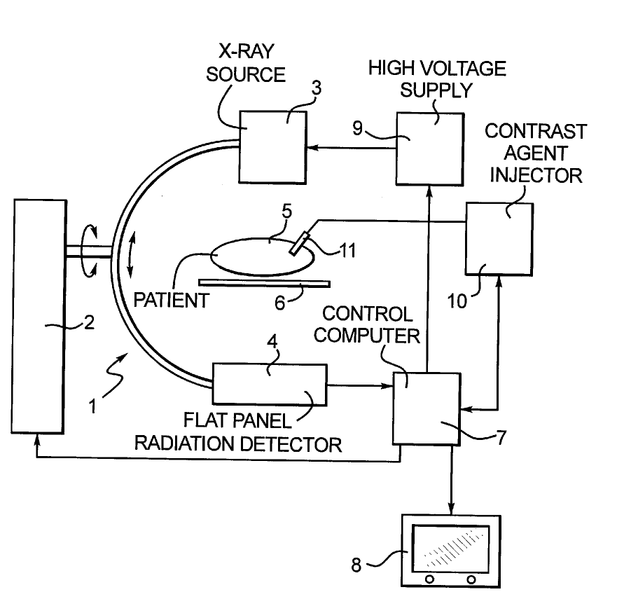

### BACKGROUND OF THE INVENTION

1. Field of the Invention

The present invention relates to C-arm computed tomography, and in particular to C-arm computed tomography for the purpose of generating angiographic images of an examination subject, with contrast agent being administered to the subject.

2. Description of the Prior Art

C-arm computed tomography is being increasingly used for the purpose of acquiring and depicting three-dimensional structures in a patient immediately before or during a minimally invasive medical interventional procedure.

The anatomical structures of interest are often vessels or other body lumens, the depiction of which in the reconstructed image requires the administration (injection) of a contrast agent to the examination subject. For this purpose, it must be ensured that, during the image acquisition, the contrast agent is present with a sufficiently high concentration in the region of the volume of interest for which three-dimensional image reconstruction is to proceed.

Conventionally, the injection of the contrast agent is usually initiated manually. The interventional radiologist must skillfully select the quantity (bolus) and the injection rate of the contrast agent so that a sufficient filling (and thus a high-contrast presentation) of the vessels of interest is achieved, while maintaining the radiation exposure of the patient at an optimally low level.

An example of a C-arm angiography system that is commercially available is the AXIOM Artis, offered by Siemens Healthcare. This system can be operated for reconstructing 3D images from 3D datasets using the DynaCT software, also available from Siemens Healthcare. In this and other angiography systems, it is important that the time interval between the injection of the contrast agent and the time of the image acquisition be such that the image acquisition occurs at a time in which the vessels in the volume of interest are optimally filled with the contrast agent.

Since many factors can enter into the progress of the injected contrast agent through the vascular system of the examination subject, it is difficult to accurately predict the point in time at which the contrast agent bolus will arrive at, and optimally fill, the vessels in the region of interest, for which the three-dimensional image is to be reconstructed. Typically, fixed protocols that are associated with each different region of interest are used to start the image acquisition at a time that is predetermined according to the protocol. This timing is embodied in the protocol based on factors such as the size and weight of the patient and, as noted above, the location of the region of interest for which an image is to be obtained. In practice, however, the predetermined timing that is embodied in the protocols results in the bolus arriving too early or too late with regard to the point in time of image acquisition, thereby resulting in a contrast in the reconstructed image that is not optimal or, in extreme cases, the reconstructed image is not even usable for diagnostic purposes, and the procedure must therefore be repeated, causing additional discomfort to the patient and resulting in increased costs.

Computed tomography systems are known that are operable in a so-called radiographic mode as well as in a CT mode. In the radiographic mode, a standard two-dimensional image of the examination subject is obtained, such as by fluoroscopy, with the C-arm being held at a stationary position relative to the patient. Such systems can be selectively switched for operation in the CT mode, wherein the C-arm rotates in the usual manner so as to obtain a large number of sets of projection data, which are used to reconstruct a three-dimensional image of a region of interest of the subject.

An example of an x=ray diagnostic device that is operable in a radiographic or radioscopic mode, as well as in a CT mode, is disclosed in U.S. Pat. No. 6,487,267. This device is disclosed in that patent as making use of gantry for obtaining the radiographic as well as the CT images, namely a gantry that rotates within a stationary frame.

A C-arm apparatus that is operable in a radiographic mode and a CT mode, particularly for angiography, is described in United States Patent Application Publication No. 2006/0120507.

### SUMMARY OF THE INVENTION

An object of the present invention is to improve the manner by which the aforementioned timing between contrast agent injection and image acquisition is determined or controlled, in the context of computed tomography. The above object is achieved in accordance with the present invention by a method and an apparatus wherein the injection of the contrast agent is monitored by operating the computed tomography apparatus in a radiographic (fluoroscopic) mode, followed by image acquisition with the same CT apparatus being operated in the CT mode. The contrast agent bolus thus can be automatically detected in the fluoroscopic image, and the course therein (filling) can be tracked. When an appropriate degree of filing of the vessels in the volume with contrast agent, which are in the region of interest for which a three-dimensional image is to be reconstructed, the acquisition of projection data for the 3D image reconstruction can be started automatically. The necessity of making a prediction, either within the confines of a protocol or by manual selection, is thus avoided.

Moreover, once the acquisition of projection data sets is automatically started, the injection rate and duration of the contrast agent that, if necessary, is continued to be administered to the patient during the data acquisition can be automatically calculated, in order to ensure that the filling of the vessels is optimal for the entire duration of the projection data acquisition.

The detection of the degree of filling of the vessels can be undertaken by subtracting respective images with and without the contrast agent therein, such as by digital subtraction angiography (DSA).

From the projection of the contrast agent bolus, the speed with which the bolus moves in the vessels of interest can be calculated. If the projection data sets are acquired using the aforementioned DynaCT system, the parameters for operating the DynaCT system for acquiring a sufficient amount of projection data sets are known, particularly the time duration for acquiring such projection data sets, so that the contrast agent injection can be stopped in an automatically time-controlled manner, so that the maximum contrast in the image is obtained with the lowest quantity of contrast agent being administered.

The aforementioned United States Patent Application Publication No. 2006/0120507 describes a system of the type commercially embodied in the DynaCT systems, and the teachings of that document are incorporated herein by reference.

The procedure according to the present invention can also be employed in the context of “classic” CT (i.e., without the use of a C-arm device). The aforementioned U.S. Pat. No. 6,487,267 is an example of such a classical CT device in which the inventive method can be employed, and the teachings of that document are therefore also incorporated herein by reference.

### BRIEF DESCRIPTION OF THE DRAWING

The single figure is a schematic block diagram of an embodiment of an apparatus constructed and operating in accordance with the principles of the present invention.

### DESCRIPTION OF THE PREFERRED EMBODIMENTS

The invention is explained in the context of the embodiment shown in the figure, on the basis of a C-arm apparatus 1. The employment of the invention, however, is not limited to a C-arm apparatus. The principles of the invention can also be embodied in a “classic” CT apparatus of the type described in U.S. Pat. No. 6,487,267, having a rotating gantry mounted in a stationary frame.

In the embodiment shown in the figure, the C-arm apparatus 1 has a C-arm stand 2 that supports an x-ray source 3 and a flat panel radiation detector 4 so as to be rotatable around a patient 5 on a patient bed 6. Such rotational capability is indicated by the circular double arrow. The C-arm apparatus 1 is also capable of orbital movement, as indicated by the curved double arrow, as well as conventional vertical movement (not separately indicated).

The C-arm apparatus 1 is operated by a control computer 7, which supplies movement signals to the C-arm stand 2 to operate motors or other movement-producing devices therein. The control computer 7 also provides operating signals to a high voltage supply 9 that, in turn, supplies appropriate voltages and currents to the x-ray source 3 for the operation thereof.

The control computer 7, in response to manual or programmed inputs, can operate the C-arm apparatus 1 either in a fluoroscopic mode, wherein the x-ray source 3 and the radiation detector 4 are held in a stationary position, or in a CT mode wherein the x-ray source 2 and the radiation detector 4 are rotated around the examination subject 5 for the purpose of obtaining sets of projection data at respective projection angles.

In both modes, the x-ray radiation from the x-ray source 3, attenuated by the patient 5, is detected by the radiation detector 4 which, in turn, supplies signals representing the attenuated radiation in both modes to the control computer 7. In the fluoroscopic mode, these signals are used to generate a two-dimensional fluoroscopy image, which is displayed on the monitor 8. In the CT mode, the projection data sets are used in any suitable, known image reconstruction algorithm to reconstruct a 3D image of a region of interest of the patient 5, which is also displayed on the monitor 8.

For simplicity, the control computer 7 is shown as serving also as an image reconstruction computer, but it is of course possible to execute the image reconstruction algorithm in a separate, specifically dedicated image reconstruction computer. It is also possible to store the output signals from the radiation detector 4 in a memory (not shown) and to reconstruct and display the image at a subsequent time. It is also possible for the data acquisition to proceed “online” and for the image reconstruction and/or image display to take place “offline” at a separate location or separate time from the data acquisition.

For examinations requiring the administration of a contrast agent, a contrast agent injector 10 is also controlled by the control computer 7, which delivers contrast agent via a schematically-indicated catheter 11 to the examination subject 5 at controlled quantity and a controlled rate.

In accordance with the present invention, an overall procedure involving the administration of contrast agent, such as for the purpose of obtaining angiographic images, is begun with the C-arm apparatus 1 being operated in the fluoroscopy mode, and the contrast agent injector 10 is either manually or under the control of the control computer 7, caused to administer a contrast agent bolus via the catheter 11 to the patient 5. With the C-arm apparatus 1 being operated to generate a fluoroscopic image at the monitor 8, the degree of filling of the vessels in the region of interest is also monitored with the control computer 7 by automatically detecting the degree of filling. When the automatic monitoring of the degree of filling indicates that an appropriate or optimal level of contrast of the vessels in the region of interest has been reached, the control computer 7 automatically switches the C-arm apparatus 1 to operation in the CT mode, and sets of projection data are obtained for reconstructing a three-dimensional image of the region of interest.

Since the time at which the contrast agent injector 10 was first actuated in order to begin delivery of the contrast agent is known, and since the time at which the optimal filling was achieved, as a result of the fluoroscopic monitoring, is also known, this patient-specific information can be used by the control computer 7 to control operation of the contrast agent injector 10 while the CT projection data are being obtained, so that the contrast agent continues to be injected (administered) at an appropriate rate and quantity for the duration of the CT data acquisition. This ensures that only as much contrast agent is administered as is necessary to continue to achieve optimal contrast of the vessels in the region of interest, without an over-administration of contrast agent.

The tracking of the contrast agent can be undertaken, for example, by subtraction angiography, in particular digital subtraction angiography (DSA).

In summary, the method and apparatus in accordance with the present invention achieve optimal timing of the image acquisition with respect to the filling of vessels of interest with contrast agent, avoid the over-administration of contrast agent to the patient, and relieve the radiological personnel from having to manually make predictions regarding the progress of the contrast agent to and in the vessels in the region of interest from which image data are being obtained.

Although modifications and changes may be suggested by those skilled in the art, it is the intention of the inventor to embody within the patent warranted hereon all changes and modifications as reasonably and properly come within the scope of his contribution to the art.

I claim as my invention:

1. A medical imaging system comprising:
an x-ray image data acquisition system configured to irradiate a subject with x-rays and to generate image data representing attenuation of the x-rays by the subject;
a control unit that operates said image data acquisition system selectively in a fluoroscopy mode, wherein said x-ray source and said radiation detector are stationary relative to the subject, to produce a fluoroscopic image of the subject, and in a CT mode, wherein at least an x-ray beam emitted by the x-ray source is caused to rotate around the examination subject to obtain a plurality of projection data sets at respectively different projection angles;
a computer supplied with said projection data sets that executes an image reconstruction algorithm, to reconstruct a three-dimensional image of a region of interest of the examination subject;
a contrast agent injector configured to interact with the subject to administer contrast agent to the subject starting from a contrast agent administration start time; and
contemporaneously with said contrast agent administration start time, said control unit operating said image data acquisition system in said fluoroscopy mode to obtain a fluoroscopic image of a region of interest containing vessels to be filled with said contrast agent and automatically monitoring the filling of said vessels with said contrast agent in said fluoroscopy mode to identify a time of optimal filling of said vessels with said contrast agent, and thereupon automatically switching operation of said image data acquisition system to said CT mode to acquire projection data sets of said region of interest with the vessels filled with said contrast agent.

2. A medical imaging system as claimed in claim 1 wherein said image data acquisition apparatus is a C-arm apparatus having a C-arm to which said x-ray source and said radiation detector are mounted.

3. A medical imaging system as claimed in claim 1 wherein said image data acquisition apparatus is a CT apparatus having a stationary frame with a rotating gantry therein, with said x-ray source and said radiation detector being mounted on said rotating gantry.

4. A medical imaging system as claimed in claim 1 wherein said control unit comprises said computer that reconstructs said three-dimensional image of the region of interest.

5. A medical imaging system as claimed in claim 1 wherein said control unit, based on said contrast agent administration start time and the time at which said optimal filling of said vessels in the region of interest occurs, controls said contrast agent injector to continue to administer said contrast agent during the acquisition of said projection data sets to avoid over-administration of said contrast agent to the subject.

6. A medical imaging method comprising the steps of:
providing an x-ray image data acquisition system configured to irradiate a subject with x-rays and to generate image data representing attenuation of the x-rays by the subject, that operates selectively in a fluoroscopy mode, wherein said x-ray source and said radiation detector are stationary relative to the subject, to produce a fluoroscopic image of the subject, and in a CT mode, wherein at least an x-ray beam emitted by the x-ray source is caused to rotate around the examination subject to obtain a plurality of projection data sets at respectively different projection angles;
supplying said projection data sets to a computer and in said computer, executing an image reconstruction algorithm to reconstruct a three-dimensional image of a region of interest of the examination subject;
operating a contrast agent injector configured to interact with the subject to administer contrast agent to the subject starting from a contrast agent administration start time; and
contemporaneously with said contrast agent administration start time, operating said image data acquisition system in said fluoroscopy mode to obtain a fluoroscopic image of a region of interest containing vessels to be filled with said contrast agent and, in said computer, automatically monitoring the filling of said vessels with said contrast agent in said fluoroscopy mode to identify a time of optimal filling of said vessels with said contrast agent, and thereupon automatically switching operation of said image data acquisition system to said CT mode and acquiring projection data sets of said region of interest with the vessels filled with said contrast agent.

7. A medical imaging method as claimed in claim 6 comprising providing, as said image data acquisition apparatus, a C-arm apparatus having a C-arm to which said x-ray source and said radiation detector are mounted.

8. A medical imaging method as claimed in claim 6 comprising providing, as said image data acquisition apparatus, a CT apparatus having a stationary frame with a rotating gantry therein, with said x-ray source and said radiation detector being mounted on said rotating gantry.

9. A medical imaging method as claimed in claim 6 comprising, based on said contrast agent administration start time and the time at which said optimal filling of said vessels in the region of interest occurs, automatically controlling said contrast agent injector to continue to administer said contrast agent during the acquisition of said projection data sets to avoid over-administration of said contrast agent to the subject.
